(19) United States
(12) Patent Application Publication
(10) Pub. No.: US 2016/0158499 A1
(43) Pub. Date: Jun. 9, 2016

(54) Two-Part Multi-Function Vascular Catheter And Integrated Circumferentially Sealing Securement Dressing

(71) Applicant: Robert E. Helm, Rye Beach, NH (US)

(72) Inventor: Robert E. Helm, Rye Beach, NH (US)

(21) Appl. No.: 15/044,994

(22) Filed: Feb. 16, 2016

Related U.S. Application Data
(63) Continuation-in-part of application No. 14/741,596, filed on Jun. 17, 2015.
Application No. 14/741,596 is a division of application No. 13/613,509, filed on Sep. 13, 2012, now Pat. No. 9,180,275.
Application No. 13/613,509 is a continuation-in-part of application No. 13/349,909, filed on Jan. 13, 2012, now Pat. No. 8,715,242.
Application No. 13/349,909 is a continuation-in-part of application No. 12/914,160, filed on Oct. 28, 2010.
(60) Provisional application No. 61/534,981, filed on Sep. 15, 2011.
(60) Provisional application No. 61/437,862, filed on Jan. 31, 2011.
(60) Provisional application No. 61/482,124, filed on May 3, 2011.
(60) Provisional application No. 61/482,564, filed on May 4, 2011.
(60) Provisional application No. 61/255,927, filed on Oct. 29, 2009.
(60) Provisional application No. 62/116,249, filed on Feb. 13, 2015.
(60) Provisional application No. 62/267,155, filed on Dec. 14, 2015.

(51) Int. Cl. A61M 25/02; A61M 39/10; A61M 39/18; A61M 25/06

(52) U.S. Cl. CPC A61M 25/02; A61M 25/0606; A61M 39/10; A61M 39/18; A61M 2025/0253; A61M 2025/028

(57) ABSTRACT

A catheter and dressing assembly is disclosed including a dressing having a distal adhesive plate configured for attachment to a skin region surrounding a catheter insertion site to provide a first circumferential seal around the catheter at the insertion site, a dressing body extending from the adhesive plate and configured to surround a catheter segment external to the insertion site and a proximal sealing element. The assembly further includes a two-part catheter having a first distal part consisting of a hub base bonded to a tubular catheter body; and a second proximal interconnector-cap that is adapted to couple to the distal hub base, whereby the attachment of the interconnector-cap to the hub-tubular body portion of the catheter establishes at least one continuous fluid pathway extending from a proximal end of the interconnector-cap to a lumen of the catheter and wherein either the hub base or the interconnector cap further comprises a mating feature for mating to the proximal sealing element of the dressing such that the dressing and interconnector-cap provide a second circumferential seal around the catheter body.

### REFERENCE TO RELATED APPLICATIONS

This application is a continuation-in-part of U.S. patent application Ser. No. 14/741,596 filed Jun. 17, 2015, which is a division of U.S. patent application Ser. No. 13/613,509 filed Sep. 13, 2012, (now U.S. Pat. No. 9,180,275 issued Nov. 10, 2015) entitled “Catheter Dressing Systems with Integrated Flushing Mechanisms,” which claims priority to U.S. Provisional Patent Application No. 61/534,981 filed Sep. 15, 2011, each of which is incorporated herein in its entirety. U.S. patent application Ser. No. 14/613,509 is also a continuation-in-part of U.S. patent application Ser. No. 13/349,909 filed Jan. 13, 2012 (now U.S. Pat. No. 8,715,242 issued May 6, 2014) entitled “Snap-Seal, Sterile, Intravascular Catheter System,” which claims priority to U.S. Provisional Patent Application No. 61/437,862 filed Jan. 31, 2011, U.S. Provisional Patent Application No. 61/482,124 filed May 3, 2011, and U.S. Provisional Patent Application No. 61/482,564 filed May 4, 2011, each of which is incorporated herein in its entirety.

This application is also a continuation-in-part of U.S. patent application Ser. No. 12/914,160 filed Oct. 28, 2010 entitled “Sealed Sterile Catheter Dressings,” which claims priority to U.S. Provisional Patent Application No. 61/255,927 filed Oct. 29, 2009, each of which is also incorporated herein by reference in its entirety.

This application also claims priority to U.S. Provisional Patent Applications No. 62/116,249 filed Feb. 13, 2015 and U.S. Provisional Patent Applications No. 62/267,155 filed Dec. 14, 2015, likewise incorporated by reference in their entireties.

### FIELD OF THE INVENTION

The present invention relates to devices and methods for providing for a sterile sealed and secure vascular catheter and access site with an easily exchangeable/interchangeable multi-function hub component, whereby exchange of the multi-function hub component does not disrupt the seal formed between the catheter hub base component and the mating/integrated sterile circumferentially sealing dressing.

### BACKGROUND OF THE INVENTION

Vascular catheters come in a variety of forms with a variety of functions. Central venous catheters, peripherally inserted central catheters (PICC), and midline catheters (collectively delineated henceforth as CPMs) are a sub group of intravenous catheters that are utilized for (1) mid and long term intravenous therapy, or (2) intravenous therapy that necessitates central administration of medications. All existing non-tunneled central, PICC, and midline catheters are single piece devices with the catheter body irreversibly attached to a single piece catheter hub and its needle or needleless connection points (tunneled catheters are a separate group of central catheters, discussed below). These catheters can be inserted using Seldinger direct peel-away sheath technique and using full CDC recommended maximum sterile barrier precautions. After placement, a securement device is typically placed (e.g. Statlock®), and then a patch type (e.g. Tegaderm®) dressing is applied over the entirety of the catheter skin insertion site, catheter hub, and catheter securement device. One to three working arms (each consisting of a segment of extension tubing attached at one end to the catheter hub and at the other to a needle or needless connectors), typically protrude from the dressing and freely dangle for use as required (e.g. intravenous fluid, hemodialysis, or medication therapy).

The only exception to the single piece design of these catheters is the group of central line catheters know as tunneled catheters, used primarily for mid and long term hemodialysis access. These catheters all have necessarily 2 or more piece designs, with separate tubular catheter and hub components. Such a separate hub and catheter mechanism is required so that the tubular catheter portion of the catheter can be tunneled beneath the skin during insertion, creating a subcutaneous distance between the skin insertion site and the vein penetration site. Such catheters are inserted surgically in the operating room, and carry a unique level of complexity and invasiveness.

In contrast to peripheral IV catheters, which have a dwell time limit of 96 hours, central, PICC, and midline catheters can be left in place for 29 days or longer. The ability for these catheters to function for such longer periods of time is often the reason that they are utilized (the other, as stated, being for delivering medication(s) to larger more central veins). Because of this prolonged dwell time, catheter and catheter access site care are particularly important, in order to limit the incidence of catheter related infection, as well as to minimize other complications such as inadvertent catheter dislodgement or loss. To combat these problems, special sterile insertion regimens (CDC and CMS mandated “Maximum Barrier Precautions”) have been developed, and ancillary care procedures such as catheter stabilization with either suture(s) or specifically designed stabilization devices (e.g. Statlock®) are routinely utilized.

Dressing care adjuncts, the purpose of which is to decrease bacterial growth at the catheter-skin insertion site, have been developed (e.g. Biopatch®), and are now routinely applied. Further efforts to decrease catheter-related infection include the use of antibiotic/antimicrobial impregnated materials (e.g. catheters, dressings). Dressing care consists of application of a CDC sanctioned patch-type dressing at the time of initial sterile catheter insertion, with subsequent removal of the entire dressing, cleansing of the catheter insertion site, and then reapplication of a new dressing at specified intervals.

The healthcare system has come under increasing scrutiny. One particular focus has been the high rate and significant morbidity, mortality and cost of catheter-acquired complications. Resulting from this has been increasing activity by healthcare regulatory bodies such as the CDC and CMS aimed at decreasing such complications. Foremost among these efforts is the reduction/elimination of vascular catheter acquired infections. The institution of Maximum Barrier Precautions during insertion is an example of this focus. Even with these efforts, long term catheter infection and loss continues. Such adverse catheter related events have been estimated to lead to the loss of 30,000 lives per year in the US alone, with costs reaching as high as 2.8 billion dollars. Such negative outcomes have become strong drivers of healthcare policy, bringing about care mandates such as withholding re-imbursement for catheter acquired infections. Catheter-related complications are clearly and appropriately in the spotlight. The following will present the multiple drawbacks and shortcomings exist in respect to currently available central, PICC, and midline catheters (CPM's) and their insertion and care regimens.

First, and one of the primary faults/deficiencies with current CPMs, is the simple fact that the catheter insertion site is not sealed and protected from the outside world. From a structural standpoint, no existing PICC, mid or central line catheter dressing provides for a full 360 degree circumferential waterproof protective seal around the catheter skin insertion site. All existing IV catheters and their associated dressings do not and cannot fully protect the IV catheter insertion site from outside contamination. In fact, with existing catheter-dressing technology—despite great effort to achieve sterile catheter insertion with maximum barrier precaution bundles (e.g. full sterile prep the insertion area, drape the entire patient's body, glove gown, cap, and mask the insertion operator)—upon removal of the drapes at the completion of the catheter insertion procedure the catheter insertion site is immediately exposed to potential outside contamination via channels created on either side of the catheter hub (by the hub's tenting up of the patch type dressing). Natural movement of the catheter and its attached extension serves to further tent the dressing upwards and sideways, leading to progressive loosening and enlargement of the channels leading directly to the insertion site. Adjunctive stabilization devices such as Statlock®, meant to stabilize and help secure the catheter, unfortunately serve to affect additional dressing tenting, and provide many non-sterile dead space areas/pockets for bacteria and other contaminants exist/propagate. Again, any movement of the catheter and/or catheter dressing serves to further loosen the patch type dressing, increasing the potential for contamination. In fact, some existing catheter-dressing structures (e.g. internal jugular vein central line catheters and their patch-type dressing) actually serve to funnel contaminants such as oral secretions directly to the insertion site. And because of its non-sealing structure, current catheter-dressing technology cannot be exposed to water, hindering showering and other patient hygienic activity without costly and time consuming additional care maneuvers (e.g. covering the extremity/area with plastic and tape). All lead to significant cost in respect to patient dissatisfaction.

In an attempt to counter the increased infectious risk associated with the use of current patch type “open” dressing technology, which allows influx of contaminants beneath the dressing, dressing adjuncts have necessarily been developed (e.g. Biopatch®, antibiotic impregnated dressings and catheters), but these are costly, and, perhaps more important, run the very real risk of further selecting out multi-drug resistant organisms (“super bugs”) through the “blanket” systematic antimicrobial use. In fact, such blanket use of antibiotics runs directly counter to infectious disease dogma. In effect, the current system operates according to a paradigm that accepts obligatory outside contamination of the catheter and its insertion site, simply relying on stop-gap measures to treat this contamination after it occurs. It would be extremely desirable to have a catheter and catheter care system that prevents contamination from occurring in the first place, by circumferentially sealing the insertion site.

Second, existing PICC mid and central line catheter and catheter dressing technology does not fully secure and stabilize the vascular catheter, leading to vessel trauma (e.g. thrombophlebitis) and premature catheter loss/dislodgement. In order to more adequately secure these long dwell time catheters, supplemental securement measures and devices such as sutures, tape, and/or Statlock®-type devices are required. The suturing of long dwell time catheters has several drawbacks, including: (1) multiple skin barrier breakpoints with quickly colonized suture material (e.g. silk), (2) pain (during placement, dwell, and removal), (3) localized skin necrosis, (4) long term scarring, (5) additional tenting upward of the patch-type dressing and creation of persistent colonized skin penetrating pockets of contamination. Drawbacks to the use of external supplemental stabilization devices (e.g. Statlock®) include: (1) the additional complexity that is added to the catheter insertion-dressing placement process, (2) additional cost, (3) additional adhesive surface area (patient discomfort), (4) additional catheter-dressing bulk, (5) additional tenting upward of the patch type dressing, (6) creation of multiple non-sterile areas beneath the tented up dressing, (7) and securement site relatively remote (upstream) from the catheter-skin insertion site, allowing for movement of the catheter between the stabilization site and the insertion site. With current stabilization technology there remains the potential for catheter dislodgement, as the catheter hub is not stabilized immediately adjacent to the insertion site.

Third, because of the non-sealing structure of existing PICC mid and central line catheter dressing technology, and the obligatory bacterial and non-bacterial contamination that universally occurs, dressing changes must be performed at specified intervals. The complex contour of the catheter and its required securement/stabilization device, combined with movement of the obligatory working extension arms, necessitates relatively frequent dressing changes. Access to the catheter is not possible for insertion site care without removing the entire dressing and its large surface area. These dressing changes necessarily involve complete removal of the existing dressing, full site re-preparation (e.g. CHG), and then application of a new dressing. This process has several drawbacks: (1) the entire dressing must be removed as there is no other way to access the insertion site (consumption of caregiver time), (2) removal of the full adhesive surface area of the dressing can be difficult/time consuming as the adhesive often stuck to the catheter and its securement device, (3) this removal is painful/uncomfortable for the patient, (4) high complexity of the dressing change procedure (difficult for patients themselves to adequately perform), (5) the complex catheter/stabilization device surface cannot be rendered sterile once initially colonized by outside contamination, (6) the required high frequency of dressing changes results in relative high hospital/caregiver inefficiency, and (7) highly variable technique leads to user-dependent highly variable result. The result of this complex process—a contaminated non-sealed IV catheter site—repeats itself over and over during the length of the catheter's dwell time.

Fourth, existing PICC mid and central line catheter technology is inflexible in respect to adjusting the catheter form to changing real time needs. This leads to the insertion of catheters with variable number of ports—this number chosen based upon prediction of access needs, before the catheter is inserted. The patient and caregiver are “stuck” with what is first put in—with current technology there is no ability to change the interactive structure/function of the catheter form to align with changing/evolving functional needs. Because one can choose to not use a port that is placed, but cannot add ports, the caregiver initially inserting the catheter is forced to estimate the numerical port needs on the high side—leading to placement typically of larger multi-port catheters (2-3 lumens). These catheters have additional bulk, etc. in respect to wearing under clothing and each port necessitates additional care. For example, if medications are to be administered 2 times per week on an outpatient basis, and a three port catheter is initially inserted, then, for the 30 day dwell time, three ports are left dangling to interfere with outpatient life activities, and to necessitate dressing changes, etc. The inability to adjust the interactive interface according to clinical and patient needs is a significant drawback to existing catheter and catheter care technology and methodology.

Fifth, with existing PICC mid and central line catheter technology, back-bleeding onto/around the insertion site frequently occurs, leading to undesirable depositing of blood at and around the catheter insertion site. Attempts to wipe away/clean this blood are typically only partially successful, leaving potent biologic culture material beneath a patch type dressing that is unable to prevent its colonization/contamination with outside organisms. Virtually all internet images of PICC lines demonstrate this blood soilage of the catheter dressing complex. Insertion technology and methodology that prevent such site contamination are clearly needed to minimize catheter related complications, and to maximize safe dwell time.

Worker/caregiver safety is a central consideration in healthcare today. The emergence of virulent and lethal blood transmitted pathogens such as Ebola, has highlighted the need to eliminate any and all healthcare worker exposure to blood and other body fluids. The existence of blood contamination at the CPM catheter insertion site is unacceptable, and yet current CPM technology cannot routinely prevent this.

The recent Ebola crisis and need to carefully control blood and bodily fluids underscores the inadequacy and breach in safety that non-sealing catheter-dressing technology provides. It has become increasingly clear that a fully sealed catheter insertion site is desired from 2 standpoints: (1) to protect the patient from outside pathogens, and (2) to protect the healthcare worker form infections harbored by the patient (e.g. Ebola virus). In a perfect world, it is desirable to have PICC, midline, and central line catheter insertion and care performed in the simplest possible way that completely protects both patients and the healthcare workers entrusted with their care. It is highly desirable to have this 2-way “blanket” protection from start to finish—from initial set-up and insertion through care and use of the catheter during its dwell time. Such 2-way full protection systems do not currently exist, as simply demonstrated by the fact that no current catheter dressing system allows the inserted catheter to get wet and still maintain safe function. The lack of seal between the insertion site and outside world allows relative free ingress (e.g. water, fluid or airborne contaminants) and egress (e.g. blood, sweat, edema fluid)—clearly unacceptable in the current era of rapidly emerging multi-resistant “superbugs” and contagious lethal viruses. Current CPM catheter and dressing approaches create an incompletely protected break in the body's normal primary protective barrier. A mechanism for separating the outside world from the inside of the patient, and for separating the outside world from materials emanating from the inside of the patient is clearly needed.

Sixth, all existing catheter insertion-dressing placement technique/methodology for CPM catheters is complex, variable, and highly operator/user-dependent. This complexity and variability in catheter insertion, catheter flushing, stabilization device placement, and dressing placement often leads to a sub-optimal and highly variable user-dependent result—increasing complications and decreasing safe dwell time.

Seventh, the sub-optimal nature of existing CPM catheter and catheter care technology leads to significant increases in health care inefficiency and cost. The initial need to use multiple separate non-integrated often compensatory components (e.g. Statlock®, Biopatch®), to the need to relatively frequently change insufficient catheter dressings (increased outpatient and inpatient nursing and equipment/supply needs), the need to apply special precautions when water exposure is anticipated, the need to replace infected, thrombotic, or non-functioning catheters, and the need to treat these catheter related complications leads to significant health care inefficiency and cost. Costs to patients such as decreased comfort, compromised outpatient quality of life attributable to catheter-dressing bulkiness/unsightliness, decreased hygiene due to lack of waterproof catheter-dressing technology, and decreased productivity must be added to these inefficiencies and costs.

Eighth, and related to the above, existing CPM catheter and catheter dressing technology is bulky, has a large adhesive surface area (painful when removed), and typically has multiple protruding parts that lead to increased patient discomfort, difficult day to day patient care, compromised activity level attributable to the cumbersome, unsightly, and non-ergonomic catheter-dressing complex catching on clothing, etc. A patient's ability to maintain normal life activity is compromised, and such activities as swimming are precluded. The obligatory dangling ports, by being prone to inadvertent pulling/disruptive forces over time, lead to an unacceptable rate of dislodgement, disruption, and loss. The ability to safely remove dangling side ports when not in use would be extremely desirable, especially during prolonged outpatient use.

Ninth, patient satisfaction/dissatisfaction has become an increasingly important issue in the healthcare system, not only because adequately meeting patient needs is correct in itself, but because of the increasingly competitive healthcare environment. Private patient rooms have become the norm, and great effort is expended in providing for an optimal patient hospital experience. Often, adverse experiences with IV (including CPM's) access are central to patient dissatisfaction. CPM catheter access that is simple, durable, trouble free, low maintenance, comfortable, and allows for normalization of patient activity to the fullest extent possible (e.g. bathing and personal hygiene) is in perfect alignment with these patient satisfaction efforts. Accordingly improved catheter dressing technology that meets these requirements is needed.

Tenth, as stated previously, existing non-tunneled PICC mid and central line catheter technology exists as a one piece non-changeable unit that is not able to be varied in its form or function over time. The particular type of catheter that is to be inserted and used (e.g. a catheter with 3 working ports) is selected prior to insertion of the catheter. Once inserted, this catheter and its chosen interaction point(s) (i.e. the hub connection points, typically consisting of one to three lines extending from the hub that each end in a needle or needleless connector) remain in place until the catheter is removed at the end of its required dwell time. What is inserted is what is used. These hub “working” extensions protrude from the placed patch-type dressing, attached to IV lines when required for medication or fluid administration, or used for direct medication injection via syringe. Because these extensions protrude beyond the dressing (“flopping in the breeze”), they are fully exposed to environmental contamination, and therefore must be thoroughly cleansed prior to each use. Furthermore these tubing extensions are bulky, and are prone to being caught on clothing, etc., increasing the rate of catheter disruption and/or dislodgement. Drawbacks to the unibody design of all existing non-tunneled catheters include: (1) bulky structure, (2) obligatory long dangling unsightly catheter extensions that can get caught on clothing, etc. (cumbersome, difficult outpatient use), (3) weight and movement of the obligatory extension lines further loosens dressing over time, leading to inadequate insertion site protection and increased need for time consuming and painful dressing changes, (4) inability to change/vary the structure of the catheter as needs change over time (e.g. if only one port is required after the first week, the three port catheter originally inserted must be left in place, with two unnecessary lines causing discomfort/inconvenience), and (5) all of these factors combining to cost and resource inefficiency as well as significant patient inconvenience and discomfort with its consequent degree of patient dissatisfaction.

The only exception to the current unibody design is the group of tunneled catheters, such as those used for long term hemodialysis access. These tunneled catheters are utilized as an alternative to the one-piece catheter (and independent/separate patch type dressing strategy) used by all other PICC, midline, and central line catheters. The underlying concept is that the one piece catheter and its patch-type dressing strategy do not adequately protect the catheter and its insertion site from outside contamination over an extended period of time. Such a one piece catheter strategy requires extensive care effort to affect adequate long term use, and even then it can result in significant morbidity and mortality—particularly with extended dwell times. It should be noted that the tunneled strategy is currently clinically applied only to central line catheters. All PICC and midline catheters employ the unibody design described previously.

The concept underlying the use of the tunneled catheter, is that of separating the catheter skin entrance site from the vein perforation site by a specified subcutaneous distance—this “tunneled” distance allowing for the body itself to provide for a degree of protection from outside contamination. The strategy is, in effect, to create a distance of subcutaneous catheter-tissue interaction that allows the body's natural protective mechanisms (e.g. immune function, scar tissue formation) to prevent skin site/outside contamination from reaching the vessel penetration site. Some tunneled catheters actually contain an integrated fibrous cuff that is meant to allow/promote tissue ingrowth in an attempt to augment the body's reactive tissue in-growth/scarring mechanism to prevent deep contamination ingress. Such cuffs also serve to help internally stabilize the catheter from such movement and/or dislodgement—this cuff is therefore a central to the form and function of these catheters. Because of the mechanism of placement, whereby the tubular portion of the catheter is first placed in the vessel and then brought out through a separate skin exit site (after being subcutaneously tunneled a distance from the actual vessel entrance point), it is required that the hub portion of the catheter—a distinct and separate and wider diameter piece—be attached to the tubular portion of the catheter using a connection device and/or mechanism. This attachment of the hub is performed only after tunneling of the separate tubular portion of the catheter is complete (as the larger diameter of the hub precludes it from being pulled through the tissue tunnel).

For all of these tunneled-type catheters, separate tubular and hub portions are necessary because of the tunneling aspect the catheter's form and function. These tunneled type catheters have 2 and even three piece designs—depending on the connection strategy used to join the tubular portion of the catheter to the hub portion of the catheter. They are structurally and functionally different from all other central, PIC, and mid line catheters in that the necessarily separate components are assembled during the insertion process, rather than being constructed as one single (joined tubular and hub portions) pre-formed piece. Also distinct from existing non-tunneled catheters, is that some of these catheters can be deconstructed and separated, and even have non-working parts exchanged after insertion is complete. This function is performed only when a non-tubular portion of the catheter has broken or otherwise failed. It is necessary for all of these catheters that an attachment mechanism be provided for that serves to connect the tunneled tubular portion of the catheter to the hub portion of the catheter. This connection is central to the form and function of the catheter, and yet it also adds complexity to the catheter insertion process, as well as potential additional points for failure. Execution of the connection is by definition user-dependent, and therefore the potential lies for connection failure and loss of the catheter, etc. While a single piece pre-connected hub and tubular portion would therefore be preferable, such a preconnected hub would not be able to be tunneled.

While the tunneled catheter strategy does offer a degree of/partial protection and stability, and decreases the reliance on patch-type dressing care to protect from infection and loss, this tunneled “protection” comes at a cost. First, the protection afforded by the tunneled strategy is incomplete, particularly in patient whose inflammatory and/or immune function is compromised, such as the immunosuppressed. The tunneled strategy relies on a vigorous reaction to the catheter, the immunosuppressed often cannot generate an adequate enough response for the tunneled catheter strategy to be effective, Second, insertion of a tunneled catheter is a surgical procedure, necessitating 2 separated incisions, and typically performed in the operating room by a surgeon—with all the attendant risks of a surgical procedure, Third, when use of the catheter is no longer required, it must be surgically removed, which can be difficult and painful—particularly for cuffed catheters where scar tissue in-growth can be quite extensive and difficult to release. Fourth, because of the complex insertion and removal process, these tunneled catheters can lead to significant permanent disfiguring scarring. So while a tunneled catheter strategy is an option for long term CPM catheter use, its form and function are not optimal, and there are clear significant downsides. An alternative to the tunneled and non-tunneled approach for central access catheters—one that is simple and maximally protects the patient from the outside world—is therefore desirable.

It should be remembered that tunneled catheters were only developed in response to the inadequacy of the one piece catheter and separate patch-type dressing approach to central line care (again, PICC and midline catheters are all unibody non-tunneled catheters)—because there was and is no perceived way to reliably seal and protect the catheter-skin insertion site from outside contamination over time. As stated previously, patch type dressing technology—that pushes the catheter hub against the skin—leaves channels on either side of the dressing that lead directly to the insertion site, which then leads directly to the vessel wall penetration site. Only by separating these sites by as much distance as possible can some degree of protection be afforded. Because of the lack of protective sealing at the skin insertion site, even cuffed catheters can become infected from ingress of outside contaminants over time, and can also be difficult and painful to remove, necessitating at least a minor surgical procedure. An alternative strategy/method to tunneling, or cuffed tunneling, is therefore required.

Some iterations of the existing 3-part central line tunneled catheter systems are designed to allow the hub portion of the constructed catheter to be exchanged should it become broken or significantly contaminated. However these systems are not meant for easy exchange of parts, and this process is complex and cumbersome (as these 3-part systems are based on “tunneled” catheter strategy, with the tubular portion of the catheter separate from the hub portion of the system, and only joined during the original surgical implant procedure). These separate catheter parts, while able to be changed should they fail, are difficult to change and require careful sterile technique by an experienced operator. The multiplicity of parts, and the complexity of “changing out” these parts by nature makes such exchanges prone to failure and poor overall outcome. Furthermore, these parts are only meant to be changed when an existing part fails, and the exchange involves replacement with an identical new part. There currently does not exist flexibility in respect to change of parts to a part with new/alternative function.

Accordingly, there is a need for catheters and catheter dressings that work in an integrated manner to improve circumferential sealing of the catheter site, improve preservation of sterility at both the insertion site and the site of attachment(s) to the catheter hub, decrease blood and/or fluid contamination at the catheter insertion site, improve catheter securement stabilization, improve catheter utility/functionality by allowing interchangeable alternative function hub interaction configurations, improve ease of use and caregiver efficiency, improve healthcare worker safety, improve patient comfort and satisfaction, simplify and standardize the catheter insertion, securement, use, and dressing placement processes, and improve safe catheter longevity/dwell time through a combination of the above attributes and improvements. Methods for optimally implementing this novel technology are also covered in this patent application.

### SUMMARY OF THE INVENTION

Methods and devices are disclosed for (1) sterilely inserting a CPM vascular catheter, (2) sterilely flushing this inserted catheter in a simple integrated fashion, (3) proximal to distal over-the-catheter hub circumferential mounting an integrated sterile-sealing dressing to maintain the sterile state achieved at the insertion site at the time of catheter insertion and to fully secure the catheter into optimal working position, (4) allowing interchanging/exchanging of the connection end (the hub “interconnector” or “working end”) of the catheter hub in order to provide for alternative use/functional status of the catheter without disrupting the integrated sterile seal between the catheter hub and the dressing, (5) allowing elimination of healthcare worker exposure to patient blood, and (6) providing for sterile preservation of the working end (distal) interconnector portion of the catheter hub through an integrated ergonomic hub cap protection device that mates with the sterile sealing dressing and/or catheter hub in sterile sealing fashion, and providing for a form-fitting overlay secondary securement device that provides for increased stability and protection.

In one exemplary embodiment, a catheter is provided that is a two-part system with several unique structural and functional characteristics. The first (distal—toward the tip of the catheter inside of the patient) “base” portion of the system consists of the tubular portion of the catheter that is permanently bonded to a hub base portion (i.e. attached during the manufacturing process so that it is packaged as a single piece sterile unit).This portion of the catheter is inserted into the patient using standard Seldinger or peel-away sheath technique. This base portion of the catheter hub has a specific circumferential mating point for the proximal (away from the patient) to distal (toward the patient) over-the-catheter mounted integrated sterile circumferentially sealing dressing. This mating point also contains an integrated “stop-flange” that precludes the catheter from being withdrawn through the mounted dressing, allowing the integrated dressing interaction to fully secure and stabilize the inserted catheter. The second portion of the system consists of a (away from the patient) hub “interconnector” that is connected to the first portion of the catheter hub through a reversible connection mechanism. This reversible connection mechanism allows removal of any given attached hub interconnector, and exchange of this interconnector for a new interconnector with the same or alternative function(s). A sealing mechanism (e.g. re-sealable split septum) in the hub base prevents backflow of blood during the insertion and/or interconnector exchange process.

In one exemplary embodiment, while the first base portion of the catheter hub is permanently attached to the tubular catheter body at its distal end, it does not on its proximal end have an attachment mechanism for attachment of standard IV lines or injection devices. Instead, it has one or more points for sterile and sealed connective interaction between the lumen(s) of the hub base (with its permanently attached/joined/bonded tubular body) and the chosen second (distal) “interconnector/cap” (known henceforth as the “IC”) portion of the hub, allowing for fluid conveyance through the base portion of the catheter hub to the IC portion of the catheter hub (and subsequently to devices attached to the IC portion of the hub). This two part catheter structure (hub base and IC) allows for the interactive function of the catheter to be changed according to patient/caregiver needs. This novel structural and consequent functional flexibility allows for adjustment of the number of functional ports, as well as the type of functional port(s). IC's with zero (i.e. simple cap), one, two, or even three or more ports can be readily reversibly attached. The clear advantage of being able to easily adjust the number of ports, or to remove dangling/bulky ports completely—while maintaining full catheter access site sterility—is currently not available (and has never been). The novel ability to completely remove the dangling/bulky hub extension/connectors that currently exist, replacing them with an IC that simply caps the system during periods of non-use, can be readily appreciated.

This two-part catheter system allows the base portion of the catheter to act as a mounting point for the sterile circumferentially sealing dressing that also acts to fully secure the catheter. The hub IC portion (the working interaction end of the catheter) is proximal to the hub base and mated dressing, thereby allowing exchange of the “working” IC portion of the catheter without disrupting the circumferential securing seal between the dressing and the hub base portion of the system.

This two-part catheter system is unique in that the dressing binds only to the hub base portion of the system, allowing free and easy exchange of the IC, while still fully preserving catheter sterility and while allowing the enhanced functional capacity readily allowed by this exchangeability. Again, the main sterile-sealing-securing dressing interacts only with the main base portion of the catheter, and not with the IC portion of the dressing. In one embodiment, the points of connective interaction between the catheter base (and its attached tubular body) and the IC can be male protrusions that insert into female receptacles in the cap portion of the hub, or the opposite. Either form has a resealable/penetrable membrane/mechanism over its proximal aspect that allows the Seldinger wire to extend through and out of the catheter hub base during Seldinger insertion, and that prevents backbleeding once the insertion wire is removed. This membrane also prevents backbleeding/insertion site soilage during exchange of the IC portion of the 2-part hub.

In one exemplary embodiment, a mechanism, method and/or system for cleaning/sterilizing/de-contaminating the surfaces of interaction between the hub base and IC portions of the catheter is provided for, so that exchange of IC's can be done with complete asepsis/sterility. Such mechanisms/methods include, for example, absorbent interactive surfaces impregnated with anti-septic solution. The IC could be pre-packaged with this attribute, or ant-septic solution can be applied to the IC interactive surface as part of the exchange process.

In one exemplary embodiment, the base (distal) portion of the catheter hub has a circumferential mating point that specifically allows for mating of a circumferentially sealing dressing. The circumferentially sealing mating point between the catheter base and the dressing is situated such that the dressing body leaves sufficient hub base protrusion proximally to allow for manual attachment of the exchangeable multi-function IC portion of the catheter hub to the base portion of the catheter hub without adversely affecting the sterile seal between the base portion of the catheter hub and the sterile sealing dressing.

In one exemplary embodiment, after mounting of the integrated sterile sealing dressing to the catheter hub base, specific grasping point on the surface of the dressing allow easy and directed manipulation of the base portion of catheter hub when performing functions such as exchange attachment of the IC component of the catheter hub. The close mating interaction between the catheter dressing and the base portion of the catheter hub allows direct grasping of the catheter dressing at these designated points to allow attachment and detachment without disrupting the sterile seal formed between the catheter hub base and the sterile sealing dressing.

In another exemplary embodiment, the base (distal) portion of the catheter hub has a distally (closest to the patients skin and the toward the catheter tip) located “stop-flange” that is larger in diameter than the remainder of the catheter hub. This stop-flange prevents “pulling out”/inadvertent removal of the catheter through the mounted dressing. From a structural standpoint this stop flange is located distal (toward the catheter tip) to the circumferential mating point. In one embodiment the stop flange consists of two winged protrusions. In another it consists of a hexagon of protruding tips. Any shape is possible with the only restrictions being that it is larger in diameter than the remainder of the first portion of the catheter hub so that the circumferentially sealing dressing can be slid down over the proximal portion of this hub base. In another embodiment, this stop-flange can form a part of the wall of the sterile chamber created by the integration of the catheter base with the integrated sterile dressing.

In another exemplary embodiment, the sterile sealed integrated circumferentially sealing dressing can be removed from the catheter, should this need arise clinically. Such removal of the dressing would involve the general steps of: (1) detaching the adhesive plate form the patient's skin, (2) grasping the dressing and catheter base, (3) providing for longitudinal force on the dressing away from the patient while stabilizing the catheter/holding in static position the hub base (with this force being strong enough to disengage the annular seal between the catheter and dressing), and (4) complete disengagement, removal and disposal of the dressing.

In another exemplary embodiment, the hub base has a proximal reversible connection point for reversible attachment of the second IC portion of the catheter hub. In one embodiment this connection consists of a snap-fit mechanism. In another embodiment this connection is of a screw-lock type mechanism. In another it is a friction fit mechanism. In another it is a combination of these reversible mechanisms.

In another exemplary embodiment, the IC portion of the two-piece hub can be constructed to provide for multiple options in respect to working end/attachment functionality of the catheter. In one embodiment this IC is a simple “non-functioning ergonomic end cap” that is used to seal the catheter hub during periods of non-use (e.g. outpatients between periodic medication injections, allowing for improved ergonomics, increased comfort beneath clothing, etc.). In another embodiment IC is formed to provide for multiple pre-attached connection lines, similar to those found in extending from existing PICC, mid and central line hubs, and each of these lines can have an attached needle or needleless connector. In yet another embodiment, the IC has one or more female connection points to which an IV line of injection syringe can be attached. In another embodiment the IC has one or more male connection points.

The IC part of the two-part catheter hub can be attached to an integrated saline flush syringe that because of its unique design (e.g. having a diameter less than or equal to the catheter base body and less than the catheter base stop-flange), also serves a second function as an integrated sterile mounting “handle”. In one embodiment this syringe is pre-attached to a specifically designed insertion cap. In another embodiment the sterile flush syringe is reversibly attached to the insertion cap, so that after catheter flushing and dressing mounting the syringe can be detached for the caps integrated needle or needleless connector. In another exemplary embodiment, the modified sterile flush syringe mounting handle has a central channel that allows passage of the wire through and out the plunger of the syringe, thereby allowing passage of the entire system over the intravascular wire during catheter insertion.

In another exemplary embodiment, the integrated sterile sealing dressing has a re-sealable access window that allows direct access to the sterile chamber formed by the mounting of the dressing to the catheter hub base and attachment of the adhesive base to the skin surrounding the insertion site. This re-sealable window allows access to and cleansing of the catheter-skin insertion site if necessary/as desired. It also allows periodic exchange of sterile chamber contents (e.g. Biopatch®) as desired. For all of these direct access functions, the need to remove the entire dressing to access and affect the catheter-skin insertion site (as with existing dressing technology) is eliminated. The sealed chamber provided for by the dressing-catheter base interaction, combined with the resealable access window, allows for long term control and maintenance of the internal milieu of the chamber.

In another exemplary embodiment, the integrated sterile sealing dressing-catheter interaction naturally provides for catheter securement (e.g. stabilization and protection against dislodgement). This integrated securement function augments—rather detracts from (as with existing securement device technology)—insertion site sterility. Current dedicated securement technology serve to (adversely) additionally “tent-up” the dressing, providing additional pockets for bacteria and other contaminants to exist/flourish.

The integrated stop-flange of the catheter base provides for both full longitudinal and rotational stability to a higher/greater degree than is currently available. Unlike existing catheter securement/stabilization technology, this stability is provided at the most distal point of the catheter hub (e.g. closest to the catheter-skin insertion site), optimizing stabilization, and providing a degree of stabilization not achievable with existing technology.

In another exemplary embodiment, an ergonomic IC protection device/cover is provided for that serves the purpose of preserving the connection points of the hub cap (the proximal portion of the two part catheter hub) in a protected/covered/sealed sterile ready-to-use state. In one embodiment this cap is designed to mate in sterile sealing fashion with the integrated sterile sealing dressing to form a single curved profile ergonomic cover that seals an ergonomically protects the inserted catheter and prevents it from catching on clothing, etc. In another embodiment, this hub cap protection device forms a sealing coupling with the portion of the base part of the catheter hub that extends form the sterile sealing dressing. In a third embodiment, the hub cap protection device mates to both the catheter hub base and the integrated sterile dressing. In one exemplary embodiment this IC protection device cover includes an antiseptic agent applied to the attachment surfaces, helping to maintain the catheter in a sterile ready to use state.

In another exemplary embodiment, a secondary activity/high risk cover is provided for that serves the purpose of covering and further sealing and securing the 2-piece catheter hub/integrated dressing/hub cap protection device. This cover form fits over the integrated catheter-dressing complex and is attached to a patient's extremity by circumferential stretchable band or an adhesive attachment plate. In one embodiment, this secondary activity cover augments the sterile seal provided for by the integrated dressing by applying directional pressure that serves to maintain and propagate adhesion of the dressing adhesive plate to the skin. In one embodiment, application of this secondary cover help dissipate/remove/protect against disruptive forces that might otherwise serve to degrade the dressing's adhesive integrity over the life of the indwelling catheter.

In another exemplary embodiment, a specific designated point along the integrated catheter dressing's interface with the base portion of the catheter hub can exert/transmit force to an integrated point of the catheter hub that serves to enact mechanical occlusion of the fluid pathway through the catheter base. This embodiment allows finger pressure to occlude to and fro fluid movement through the catheter when desired, such as when exchanging catheter hub caps, or to prevent backbleeding. In one embodiment, finger pressure at the designated point on the dressing transmits this force to an inner protruding ridge. This inner protruding ridge than transmits the force to an integrated soft point in the bub cap base, that then effects compression of the catheter lumen (while finger pressure is applied). When finger compression pressure is released, the catheter lumen returns to (non-occlusive) normal form and function.

In another embodiment, a method for inserting a 2-piece catheter and integrated sterile sealing dressing is described, whereby first a guidewire is inserted using standard maximum barrier precautions (MBP) and standard Seldinger technique. The base portion of the catheter hub is then threaded over the wire and into the patient to the desired depth, with the guidewire pushed through the resealable membrane covering the chosen male (or female) end of the catheter base. The guidewire is then removed. The hub cap with its attached saline flush device/mounting handle is then attached to the catheter hub base, and the saline flushed into the inserted catheter. The circumferentially sealing dressing is then mounted over the saline flush “handle “and slid down into mating position on the catheter hub base and the base adhesive plate applied. The sterile saline flush handle is then removed. The chosen IC is then attached. A hub cap protection device can then be placed as outlined below [23]. In one embodiment, the sterile flush “syringe” mounting handle can be detached from its component IC, and the IC simply left in place as an IC. In another embodiment, the flush handle is formed as a single piece unit with an integrated IC-type component, and this single unit is removed after flushing, and simply replaced with a designated/separate IC.

In another exemplary embodiment, the method for exchanging IC's (old to new) is provided. First the designated grasping points on the integrated dressing are grasped with two or more fingers to stabilize the base portion of the catheter hub (Note: this designated point may also be the point containing the reversible lumen occlusive mechanism delineated in [20]). The IC portion or the hub is then detached (by its reversible mechanism). The new IC is then attached (by the same reversible mechanism), flushed in standard fashion, and utilized as desired.

In another exemplary embodiment, the method for placing an ergonomic secondary sterile sealing IC protection device/cover is provided, whereby the secondary IC protection device is reversibly attached in sealing engagement to the integrated dressing, the IC, and/or the catheter hub base during periods of non-use.

In another exemplary embodiment, the method for changing the integrated sterile sealing dressing is provided (should this be required). First, the adhesive plate if circumferentially detached from the skin. Next, the dressing is pulled/slid back over the two piece hub, disengaging it from its mating point on the catheter hub base. After thorough cleansing/re-sterilization of the catheter hub and skin insertion site, a new sterile dressing is applied as described previously. This process can be aided by attachment of a new sterile saline flush mounting handle.

In another exemplary embodiment, the method for accessing the sterile chamber through its re-sealable access window is described. First, the re-sealable access window is opened. The contents/status of the sterile chamber is then effected as desired (e.g. new Biopatch® exchange). After this process is complete, the re-sealable access window is closed/re-sealed.

In another exemplary embodiment, the method for maintaining the sterility of the sterile chamber of the integrated catheter dressing is provided. According to this method, a sterilizing agent (e.g. liquid, gas, anti-microbial sponge, and/or other anti-microbial containing body) is injected or inserted through the re-sealable access portal at specified/desired intervals).

In another exemplary embodiment, the two mating interaction points of the catheter and dressing (the stop flange and annular recess and their respective counterparts on the dressing), form a sealing engagement that prevents both withdrawal of the catheter from the mounted dressing, as well as removal of the dressing from the mated catheter hub base. A releasable mechanism is provided for that allows the mounted integrated dressing to be removed from the in-dwelling catheter should this be required.

In another exemplary embodiment, the hub base and hub cap mate by a detachable mechanism formed by an annular snap fit sealing engagement, the proximal wall of the annular depression and its matching annular ring projection of the dressing sloping in a proximal (away from the catheter tip and patient body) direction as they deepen toward the longitudinal center of the catheter hub, so that once engaged in snap-fit mating fashion, the hub cap cannot be removed without specified intentional directed force aimed at disengaging this interaction. This force could be derived from finger depression pressure elsewhere on the hub cap (remote from the annular projection) so that a lifting outward force is generated at the mating point when this remote point is depressed. The force transfer from the finger depression point to the annular mating point could be effected by a relatively stiff lever arm(s) within the body of the dressing at this point.

### BRIEF DESCRIPTION OF THE DRAWINGS

The invention will be more fully understood from the following detailed description taken in conjunction with the accompanying drawings.

FIG. 1 is a top view of one embodiment of a two piece exchangeable catheter design.

FIG. 2 is a top view of another embodiment of a two piece exchangeable catheter with a saline flush syringe reversibly attached to a hub cap section of the hub.

FIG. 3 is a top view of another embodiment of a two piece exchangeable catheter and a hub cap having snap-fit receptacles for any lines or injection devices to be attached for use.

FIG. 4 is a top view of another embodiment of a two piece exchangeable catheter and tubing extensions that are fixedly attached/bonded to the catheter hub cap.

FIGS. 5A-5I present a series of views that illustrate the step by step process of two piece catheter hub Seldinger insertion, saline flush/hub cap attachment, saline flushing of the catheter, dressing mounting over the saline syringe mounting handle, removal of the syringe/mounting handle, and then attachment of a fluid line, or optionally, placement of the hub cap sterility preserving protection device.

FIG. 6A is a top view of an embodiment of the hub base with the mounted integrated fully sealing and securing dressing.

FIG. 6B is a side view of the embodiment depicted in FIG. 6A.

FIG. 7A is a top view of an embodiment of a two-lumen catheter base with a zero lumen hub cap.

FIG. 7B is a side view of the embodiment of the two lumen catheter version of the integrated device depicted in FIG. 7A.

FIG. 8A is a top view of an embodiment of a two lumen catheter base with an ergonomic sterility preserving hub cap protection device in place over the zero-lumen hub cap.

FIG. 8B is a side version of the embodiment depicted in FIG. 8A.

FIGS. 9A and 9B illustrate an embodiment of a secondary activity support cover (top and side views, respectively) that form-fit to the catheter hub and hub cap protection device to stabilize and further protect the catheter during times of increased activity.

### DETAILED DESCRIPTION

In one aspect of the invention, a two-piece catheter is disclosed having a first distal part consisting of a hub base bonded to a tubular catheter body; and second proximal interconnector-cap that is adapted to couple to the distal hub base, whereby the attachment of the interconnector-cap to the hub-tubular body portion of the catheter establishes at least one continuous fluid pathway extending from a proximal end of the interconnector-cap to a lumen of the catheter. The hub interconnector-cap can have multiple lumens that reversibly connect to the multiple lumens of the hub base portion of the catheter.

In one embodiment of this two-piece catheter, the hub base lumen(s) can join to the interconnector-cap lumen(s) through a male to female joining or by any mechanism that established sealed continuity between the fluid pathway(s) of the hub base and the fluid pathway(s) of the interconnector-cap. For example, the body of the catheter hub base can join to the catheter interconnector-cap by a sealing reversible coupling mechanism, such as snap-fit lock or friction fit. The joining of the body of the catheter hub base to the catheter interconnector-cap can effect a circumferential seal by a sealing reversible coupling mechanism. Preferably, the hub base and interconnector-cap mate by a detachable/reversible mating mechanism. This mechanism provides clinically adequate integrity of the base portion and interconnector-cap portion of the hub when the two are in the attached state.

In another embodiment, the hub base and hub interconnector-cap can be joined by a snap-fit mechanism formed by an annular snap fit sealing engagement, the proximal wall of the annular depression and its matching annular ring projection of the dressing sloping in a proximal (away from the catheter tip and patient body) direction as they deepen toward the longitudinal center of the catheter hub, so that once engaged in snap-fit mating fashion, the interconnector-cap cannot be removed without specified intentional directed force aimed at disengaging this interaction. This force could be derived from finger depression pressure elsewhere on the interconnector-cap (remote from the annular projection) so that a lifting outward force is generated at the mating point when this remote point is depressed. The force transfer from the finger depression point to the annular mating point could be effected by a relatively stiff lever arm(s) within the body of the dressing at this point.

Additionally, a secondary attachment mechanism, aimed at supporting an annular attachment mechanism can be provided between the hub base and interconnector-cap. This secondary reversible attachment mechanism can provide additional integrity by the reversible bond between the catheter hub base and the interconnector-cap. The reversible secondary attachment mechanism can be chosen from a group that includes, snap fit, locking arm, magnetic, screw, bayonet, screw lock, or any other joining mechanism that meets the requirement of reversibly and stably attaching the hub base to the interconnector-cap.

In another aspect of the invention, the male or female lumen connection point of the hub base can be covered by a reversible sealable penetrable membrane/mechanism designed to allow penetration of the insertion guidewire, but to prevent back-bleeding from the catheter around this wire or through the connection point after wire withdrawal.

In yet another aspect of the invention, the hub base can have a specific circumferential mating point for a circumferentially sealing a dressing that provides sterile isolation of the catheter when inserted into a patient. This mating point can consist of an annular ring depression, or other snap-fit type engagement point. The dressing is configured to provide a first sterile seal to a patient's skin at an insertion site and a second circumferential mating seal around the catheter hub base to effectively isolate the catheter from pathogens or foreign matter. In certain embodiments, the dressing includes an adhesive plate for attachment to a patient's skin around a catheter insertion site. Attachment of the dressing's adhesive plate around the catheter-skin insertion site creates a first circumferential seal and joinder of the dressing and the catheter hub base creates a second circumferential seal.

To form the second circumferential seal, a annular mating point of the catheter hub base can include a profile that once the dressing is mated at this point, and the dressing-catheter annular mating point interaction prevents unintentional detachment of the dressing from the mating point of the catheter. One example of this would be that the proximal annular indentation in the wall of the hub base is vertical or slopes proximally (as it deepens and forms the proximal wall of the annular recess, proximal being away from the catheter tip/patient). A matching mirror image shaped catheter dressing mating projection is then able to be snapped into position at this annular mating point on the catheter, and so that the mated dressing cannot be pulled back proximally because of this formed mating interaction.

Thus, the first and second circumferential seals define a sterile chamber that surrounds the catheter-skin insertion site and the distal portion of the hub base/proximal catheter body. The dressing body can further comprise a re-sealable access portal or window that allows adjustment of the internal milieu of the sterile chamber formed by the sealing catheter-dressing-skin interaction. In certain embodiments, the proximal exit/emergence site of the catheter hub base from the dressing is substantially perpendicular to the skin insertion site opening of the dressing (i.e. parallel to the skin surface).

In other embodiments, the hub base can include a secondary mating and catheter stabilization point consisting of a stop flange of a greater diameter than the remainder of the hub base, so that this stop flange mechanically prevents withdrawal of the catheter from the integrated circumferentially sealing dressing once this dressing is mounted and mated with the catheter hub base. For example, the mating interaction points of the catheter and a dressing (the stop flange and annular recess of the catheter and their respective counterparts on the dressing), can form a sealing engagement that prevents withdrawal of the catheter from the mounted dressing, as well as removal of the dressing from the mated catheter hub base.

The catheter hub mating point can work in conjunction with the stop flange of a dressing to seal and secure the inserted catheter once this catheter is mated to the integrated sterile sealing dressing, such that when the dressing is mounted to the catheter hub base in mating fashion, the stop flange forms a portion of the wall of the created sterile chamber.

In yet another aspect of the invention, sterile kits are disclosed that include the two-part catheter of the present invention and a dressing as described above.

The hub interconnector cap can have multiple configurations, including but not limited to a simple end cap without interactive connectivity, or hub caps with one or multiple points of interconnectivity. These interconnection points be either needle or needleless in form, and can include extension tubing or other standard variations. These interconnection points, once attached via the hub cap, can be in fluid pathway continuity with the hub cap, hub base, and tubular portions of the catheter. The catheter hub interconnector cap can be detached from the hub base and replaced with a hub cap of the same or different fluid pathway number or other configuration.

In yet another aspect of the invention, methods of deploying a two-part catheter are disclosed with a two-piece exchangeable hub, whereby the interconnector cap can be removed from the hub base and exchanged with another interconnector cap with the same or different interactive configuration. For example, the method can include the steps of: (1) uncoupling the secondary interconnector cap to hub base attachment mechanism (2) uncoupling the male-female lumen sealing attachment point to the hub base (and (3), removing the first interconnector cap, and then attaching a new interconnector cap with the new desired configuration (e.g. change a 2 port interconnector cap to a simple sealing interconnector cap). The mating surfaces of the hub interconnector cap and hub base can also be covered with an anti-microbial agent so that the joining of the interconnector cap to the hub base is rendered sterile.

The catheter interconnector cap can also be reversibly attached a specifically designed catheter flush containing body/syringe that serves to (1) integrally flush the newly inserted catheter, (2) act as a mounting handle over which a sterile sealing dressing can be slid from proximal to distal.

The sterile flush syringe can have an outer diameter less than or equal to the body of the interconnector cap and hub base of the catheter of claim 1, and less than the diameter of the stop flange, as described above, of this catheter. The sterile flush syringe can flare at its distal end to the diameter of the hub cap so that the dressing can be easily slid/transferred form the syringe body to the hub interconnector cap during mounting. In one embodiment, the insertion portion of the interconnector cap (with its attached flush syringe) can be in fluid continuity with all of the fluid pathways of the chosen hub base when attached, so that all lumens of the base will be flushed with saline by injection of the saline contained in the saline flush syringe.

The catheters of the present invention can be inserted into the patient blood vessel using peel-away sheath insertion technique, whereby the catheter hub base and catheter interconnector cap complex is inserted through the previously positioned peel away sheath and into final position. The catheter hub base, catheter hub interconnection cap, and a catheter flush body/mounting handle can be preassembled as a single reversibly interconnected unit that can be inserted into the patient using through the peel-away sheath technique. For example, the tubular portion of the catheter, the hub base, the hub interconnector cap, and the attached insertion flush containing body/syringe complex can be passed over the insertion wire in Seldinger fashion to pass through the skin insertion site and into the blood vessel to the appropriate final position. After confirming optimal position, the wire is withdrawn from the assembly. In this embodiment, the insertion portion of the hub interconnector cap and its attached flush containing body/syringe can have a central channel that allow free through passage of an insertion wire. Alternatively, the insertion wire can be formed as an integral part of the catheter flush containing body/mounting handle and used for self-contained over the wire insertion of the catheter.

After insertion of the catheter assembly, saline contained in an attached flush containing body/mounting handle can be injected into the catheter hub interconnector cap, base, and tubular portion of the catheter to effect flushing of the lumen(s) of the contiguous system. After insertion and flushing of the catheter, the saline syringe and hub interconnector cap are used as a mounting handle to facilitate the sliding the mating and sealing dressing downward and into final integrated mating position on the hub base.

During period of non-use, a catheter hub interconnector cap without external fluid channel interaction point(s) can be chosen and attached, to preserve the catheter of the present invention in a ready-to-use sterile state with optimal ergonomic form/shape so that catching on things such as clothing is minimized. Additionally, an ergonomic secondary hub interconnector cap protection device can be attached in sealing and engaging fashion with the hub cap and/or dressing so that a smooth ergonomic arc is completed to further minimize catching on clothing, etc. Moreover, an accessory form fitting activity cover can be attached as a protective form-fitting cover over the entire catheter-dressing complex, acting to further seal and secure the catheter. The accessory form fitting activity cover can act as a protective form-fitting cover over the catheter-dressing complex, acting to further seal and secure the catheter, but which allows protrusion of the working ends of the interconnector cap from the secondary cover through dedicated exit points while movement of the catheter with respect to the patient does not disrupt a seal formed between the sheath and the catheter.

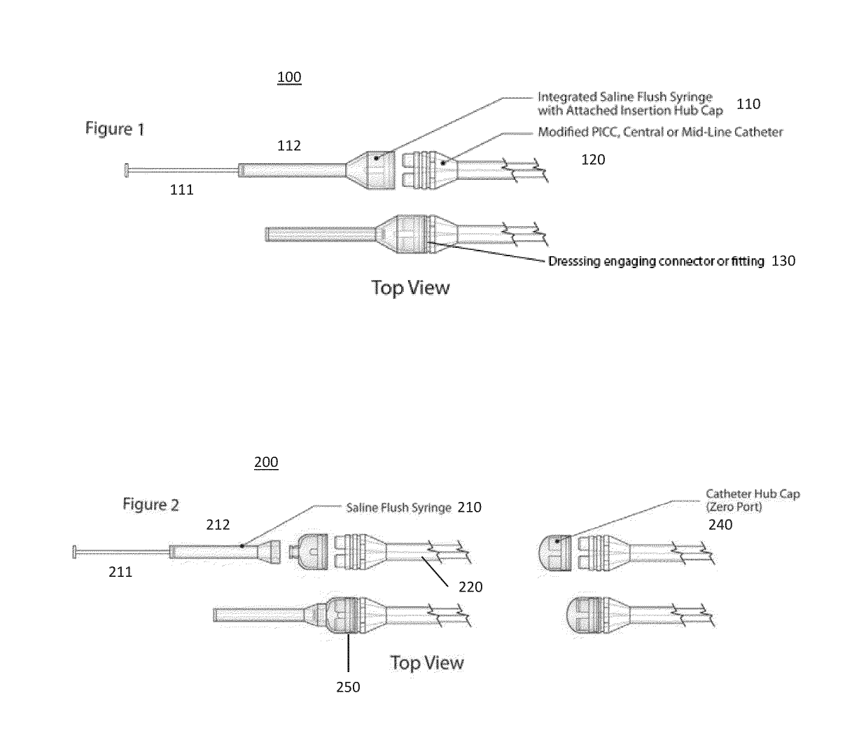

FIG. 1 is a top view of one embodiment of the two piece exchangeable catheter design 100. Shown are the base portion of the catheter which includes a joined tubular catheter body and hub base 120 (right hand side of the drawing, clear/white color), and the mounting cap embodiment of the second or “cap” portion of the catheter hub 110 (left hand side of the drawing, blue color). This version of the hub cap 110 has an integrated saline flush/mounting handle syringe 111/112. Using this embodiment, after saline flush and dressing mounting, this cap (and integrated syringe) is discarded and the desired cap (e.g. 2-port cap) reversibly attached to the base section. Notable in this view are: (1) the mounting site 130 on the catheter hub base which includes an annular recess where the corresponding annular ring protuberance on the dressing mounts and seals, and (2) hexagonal stop flange that is larger in circumference than the remainder of the catheter hub so that the catheter cannot be withdrawn through the central channel of the catheter dressing, (3) the male projection(s) extending from the base portion of the catheter hub over which the hub cap will mount to form a continuous fluid pathway between the two hub cap portions, (4) the reversibly sealable membranes over these male projections that allow penetration of the insertion wire during Seldinger insertion (but prevent backbleeding when this wire is withdrawn, and/or during hub cap exchanges), (5) the plunger 111 of the saline syringe 110 shown both in its initial pre-injection/full state and in its flushed ready-to-mount (with the integrated dressing) state. (The terms “hub” and “hub base” are used interchangeably herein unless the context would dictate otherwise. Generally, speaking a hub base refers to any type of catheter hub that can be joined to a cap element as described herein.)

FIG. 2 is a top view showing a related embodiment which features a saline flush syringe 210 that is reversibly attached to the hub cap section of the hub 250 so that the cap can be left in place after catheter flushing and dressing mounting. After mounting of the dressing the saline mounting handle syringe is detached and discarded. Two key features of the saline flush syringe 210 are depicted: (1) a maximum diameter that is less than or equal to the catheter hub mounting point, and less than the catheter hub base stop flange, and (2) the flared distal end that provides for simple and smooth guided transition from the syringe to the catheter hub base during dressing mounting. The two drawings on the right hand side show a simple “zero-port” cap 240 that can be exchanged for the initial syringe mounting cap. It is this zero-port cap 240 that would be placed prior to prolonged periods of non-use (e.g. between intermittent medication injection during outpatient treatment). Also depicted in this embodiment is the reversibly sealable needle or needleless (male or female) connection point on the insertion hub cap 250 that is in fluid pathway/channel continuity with the continuous channel formed by the catheter body and its conjoined hub base and the hub cap 250.

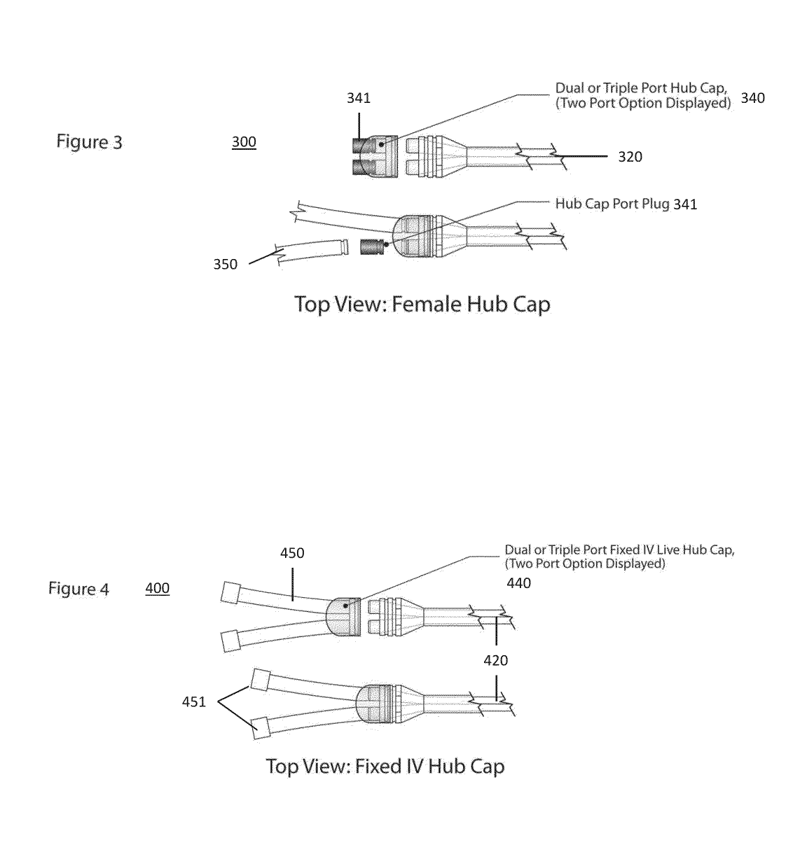

FIG. 3 is a top view showing another related embodiment whereby the hub cap 340 has the form of snap-fit receptacles 341 for any lines or injection devices 350 that are to be attached for use. Pictured is a generic depiction of this attachment mechanism. Each attachment portal is able to establish continuity with the fluid channel(s) formed within the catheter body 320, hub base, and hub cap 340. The attachment(s) can be effected by any mechanism currently in general clinical use.

FIG. 4 is a top view of another related embodiment whereby tubing extensions 450 are fixedly attached/bonded to the catheter hub cap 440 (similar in form to the net result of traditional one piece PICC, mid, or central line catheters). The proximal end of each of these fixed lines typically has an attached needle or needleless connector 451 (depicted by the small clear/white boxes on the left side of the drawing). Note that the 2 port cap 440 pictured here can be exchanged for any other desired configuration ranging from a zero port “storage” simple cap, to a 2-4 multi-lumen cap, depending on the clinical need.

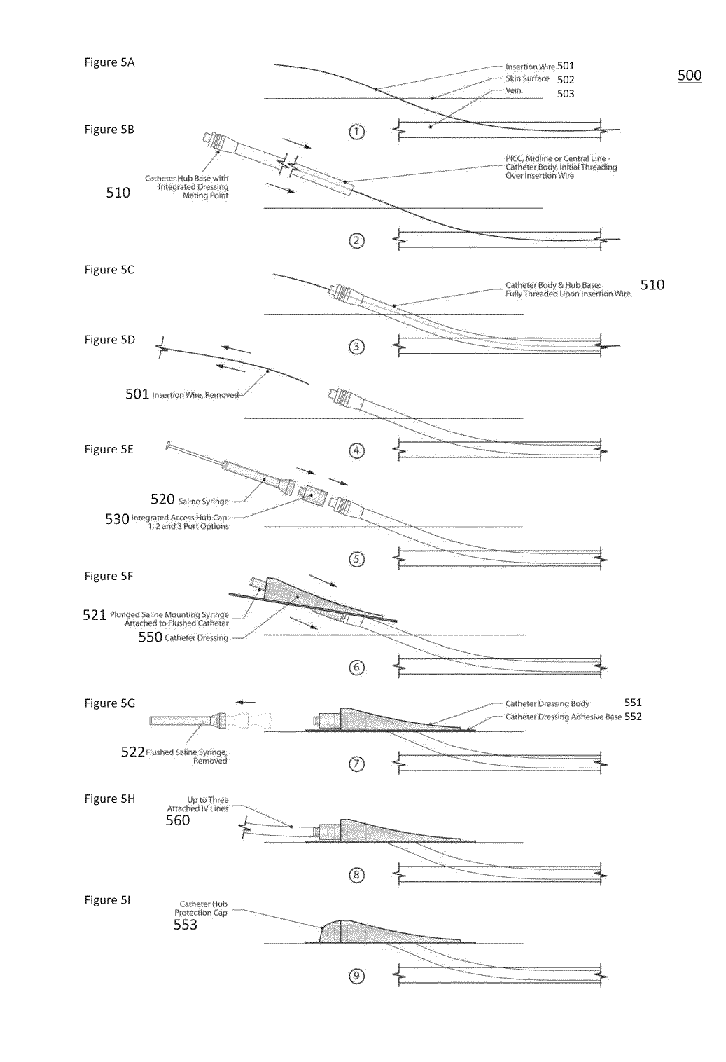

FIGS. 5A-5I present a series of views that demonstrate the step by step process of two piece catheter hub Seldinger insertion, saline flush/hub cap attachment, saline flushing of the catheter, dressing mounting over the saline syringe mounting handle, removal of the syringe/mounting handle, and then attachment of a fluid line, or optionally, placement of the hub cap sterility preserving protection device. Details of this insertion/dressing placement 500 and use process are as follows (FIGS. 5A-5I):

FIG. 5A depicts percutaneous insertion of the catheter guidewire 501 into the target blood vessel 503.

FIG. 5B depicts sliding the one piece catheter body and hub base 510 over the guidewire 501.

FIG. 5C depicts catheter inserted to final/desired insertion point/depth. Evident is the insertion wire protruding through the male extension (and its re-sealable membrane) of the hub base.

FIG. 5D shows removal of the insertion wire 501.

FIG. 5E demonstrates attachment of the saline flush mounting handle 520 and hub interconnector-cap 530 (note that these can be packaged as a pre-assembled unit, or they can be joined together on the sterile field) to the catheter hub base.

FIG. 5F depicts (1) the flushed catheter (with the plunger of the syringe 521 fully depressed), and (2) the integrated sterile sealing dressing (green) 550 being mounted over the syringe mounting handle. The diameter of the syringe mounting handle (i.e. less than the catheter hub base stop flange) should be noted, as well as the flaring outward at the distal end of the syringe to allow smooth mounting transition from the syringe to the hub base.

FIG. 5G demonstrates the sterile sealing dressing in its final mounted position. The syringe mounting handle 522 has been detached (left side of drawing), leaving the hub cap component of the 2-piece hub in place, with its protruding needle or needleless connector(s). The adhesive base plate 552 has been attached to the skin circumferentially around the catheter-skin insertion site. The mating seal between the dressing and the catheter hub base 552 has been effected. The stop flange component of the hub base 552 has been engaged, preventing withdrawal of the catheter from the attached dressing. The only way that the catheter can be removed is to remove the entire adhesive base plate 552 dressing from the patient's skin. Alternatively, the mating seal can be between the dressing and a interconnector-cap.

Note that a version of Seldinger type insertion can be practiced as an alternative, whereby the insertion wire is passed through a continuous channel that exists in the hub base, hub cap, and attached specially constructed insertion syringe (wherein the syringe has a central channel that allows through egress of the insertion wire), so that the entire catheter and attached mounting handle syringe are passed over the insertion wire.

FIG. 5H demonstrates attachment of an IV line 560 to the needle or needleless connection port.

FIG. 5I depicts placement of the sterility preserving ergonomic hub cap protection device 553, that serves to ergonomically protect the connection point of the hub cap (e.g. prevent catching on clothing), and also to maintain it in a continuous sterile ready-to-use state.

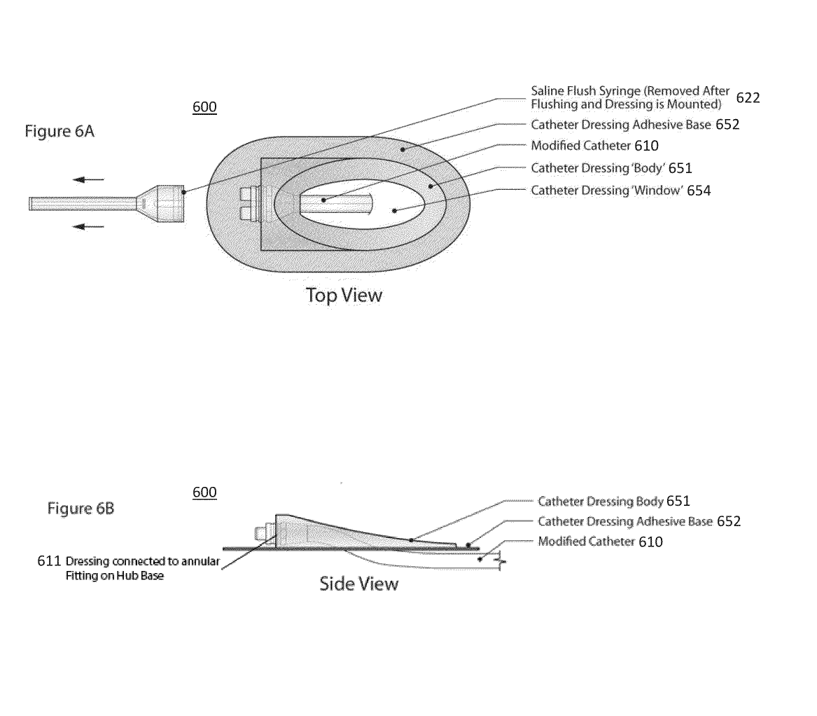

FIG. 6A shows the hub base with the mounted integrated fully sealing and securing dressing 600. The insertion saline flush device 622 and insertion cap have been removed prior to attaching the desired hub cap (e.g. zero-lumen, triple-lumen). Back-bleeding is prevented by resealable membranes covering the male ends of the hub base fluid pathways.

FIG. 6B shows a side view of the same (mounted integrated fully sealing and securing dressing 600). The dressing 600 can be slid over the catheter 610 with its integrated hub base 652 to form two sterile seals. The first seal is formed around the catheter insertion site, where the catheter penetrates through the skin. In one embodiment, this first seal is formed by the adhesive that binds the base to the skin, encircling the insertion site and preventing contamination via the skin surface. The second seal 611 is formed when the proximal end of dressing slides over the catheter hub base and connects to a mating feature on the catheter hub base. For example, the second seal 611 can be formed by an annular connector or fitting, e.g., either a circumferential rim or groove on either the catheter hub or the dressing (and a mating feature on the other) such that the dressing can mate with a corresponding feature and “snap-fit” onto the catheter hub base. Alternative, the annular fitting can take the form of matching circumferential grooves on both the dressing and the catheter hub with one or the other further including an “O” ring or other gasket material. Various further designs will be apparent to those skilled in the art so long as they suffice to form a second seal that prevents contaminants from penetrating via the catheter, thereby creating a complete sterile seal around the inserted catheter.

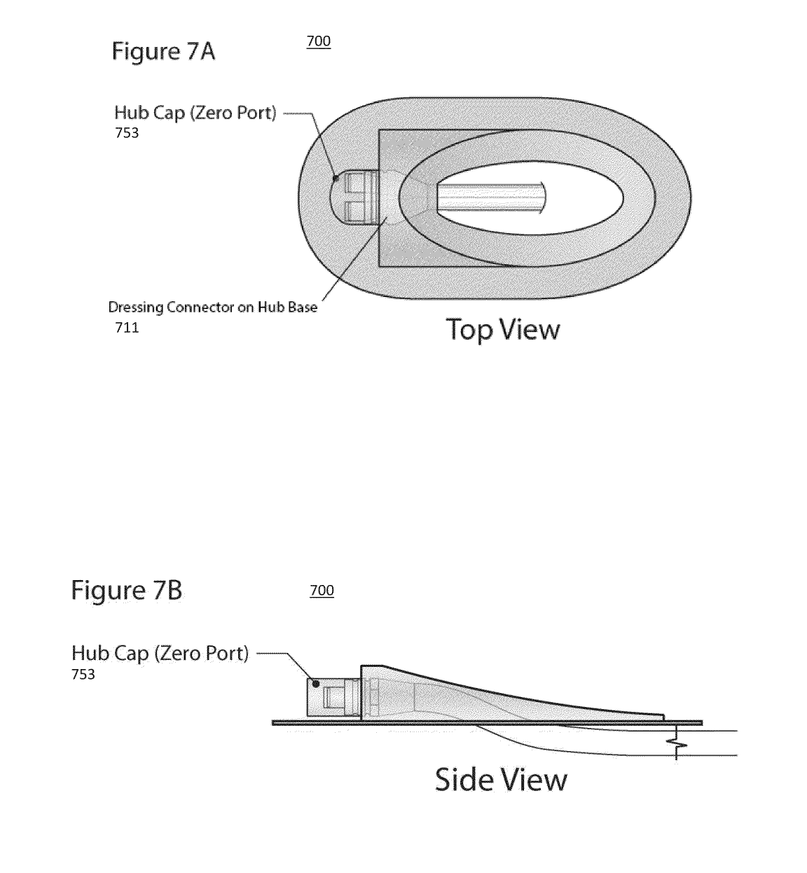

FIG. 7A shows a bird's-eye view of a two-lumen catheter base with a zero lumen hub cap 753 for use during times of catheter non-use (e.g. as an outpatient between treatments) treatments supplementary supportive activity cover for use during times of catheter non-use.

FIG. 7B shows a side view of the two catheter version of the integrated device 700 depicted in FIG. 7A.

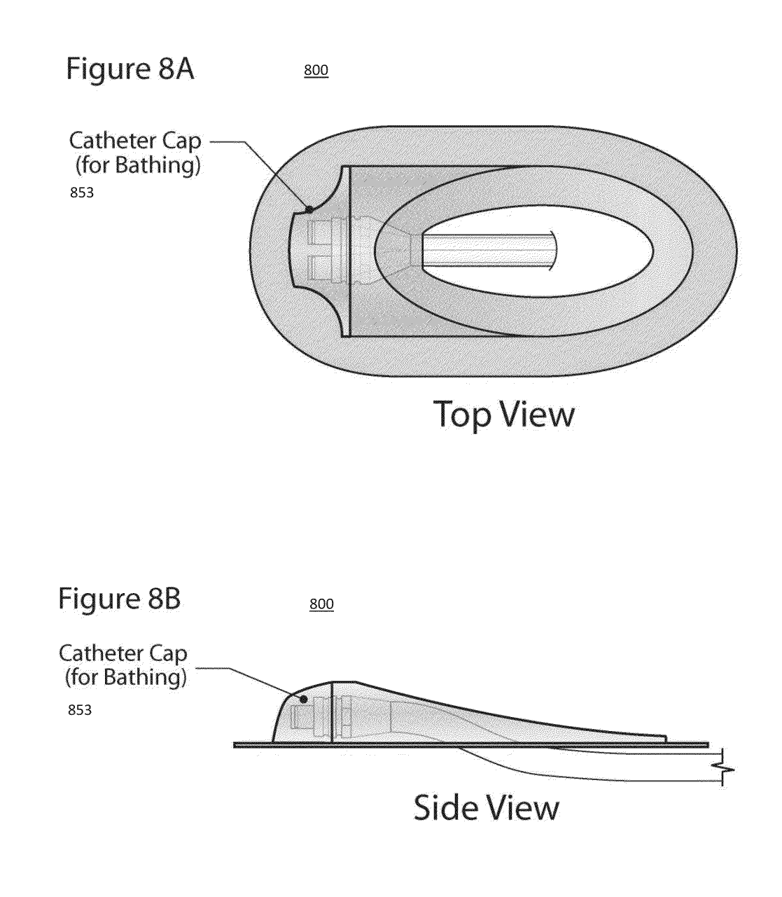

FIG. 8A shows a bird's-eye view of a two lumen catheter base 800 with a zero lumen hub cap with an ergonomic sterility preserving hub cap protection device 853 in place over the zero-lumen hub cap. This hub cap protection device 853 serves to complete an ergonomic arc to improve patient comfort during times of non-use, helping to prevent catching on clothing etc. This hub cap protection device 853 also forms a second seal against the outside world, safely allowing such activities as bathing, showering, and swimming without other preparation.

FIG. 8B shows a side version of the hub cap protection device 853 in place over the zero-lumen hub cap.

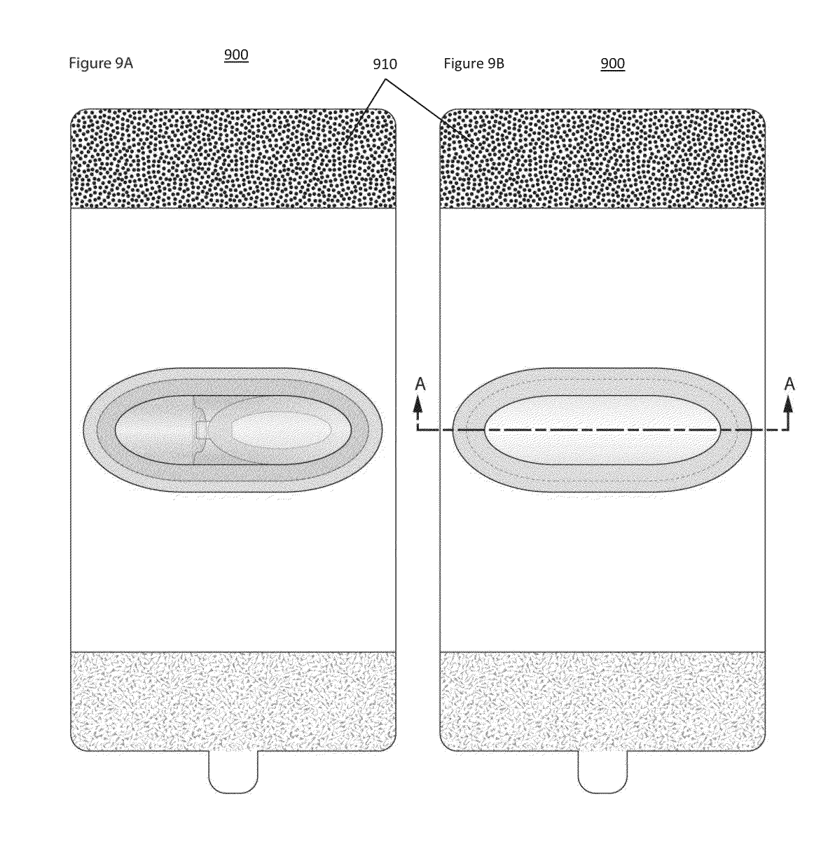

FIGS. 9A and 9B shows the secondary activity support cover 910 (top and side views, respectively) that form-fits to the catheter hub and hub cap protection device 900 to stabilize and further protect the catheter during times of increased activity. This would be particularly important for chronic outpatient use, of for use in the very young or confused patient. This support cover could have built-in egress points to allow use with a hub cap with attached extensions/lines.

Further aspects of catheter sealing and flushing techniques useful in the practice of the inventions disclosed herein can be found in related patent applications, such as U.S. patent application Ser. No. 12/914,160 filed on Oct. 28, 2010 entitled Sealed Sterile Catheter Dressings; U.S. patent application Ser. No. 13/349,909 filed Jan. 13, 2012 entitled Snap-Seal, Sterile, Intravascular Catheter System and U.S. patent application Ser. No. 13/613,509 filed Sep. 13, 2012 entitled “Catheter Dressing Systems with Integrated Flushing Mechanisms, each of which is incorporated herein in its entirety.

What is claimed is:

1. A catheter and dressing assembly comprising:
a dressing comprising
a distal adhesive plate configured for attachment to a skin region surrounding a catheter insertion site to provide a first circumferential seal around the catheter at the insertion site,
a dressing body extending from the adhesive plate and configured to surround a catheter segment external to the insertion site and
a proximal sealing element; and
a two-part catheter comprising
a first distal part consisting of a hub base bonded to a tubular catheter body; and
a second proximal interconnector-cap that is adapted to couple to the distal hub base, whereby the attachment of the interconnector-cap to the hub-tubular body portion of the catheter establishes at least one continuous fluid pathway extending from a proximal end of the interconnector-cap to a lumen of the catheter
wherein either the hub base or the interconnector cap further comprises a mating feature for mating to the proximal sealing element of the dressing such that the dressing and hub-base or interconnector-cap provide a second circumferential seal around the catheter body.

2. The catheter and dressing assembly of claim 1, whereby the interconnector-cap has multiple lumens that reversibly connect to multiple lumens of the hub base portion of the catheter.

3. The catheter and dressing assembly of claim 1, whereby the body of the catheter hub base additionally joins to the catheter interconnector-cap by a sealing reversible coupling mechanism, such as snap-fit lock or friction fit.

4. The catheter and dressing assembly of claim 1, whereby the hub base comprises a reversibly sealable/penetrable membrane designed to allow penetration of the insertion guidewire, but to prevent backbleeding from the catheter around this wire or through the connection point after wire withdrawal.

5. The catheter and dressing assembly of claim 1, whereby the hub base has a specific circumferential annular fitting for a circumferentially sealing to the dressing.

6. The catheter and dressing assembly of claim 5, wherein the mating feature of the hub base comprises an annular groove and the sealing element of the dressing comprises an annular ridge adapted for snap-fit engagement with the annular groove of the hub base.

7. The catheter and dressing assembly of claim 5, wherein the mating feature of the hub base comprises an annular ridge and the sealing element of the dressing comprises an annular groove adapted for snap-fit engagement with the annular ridge of the hub base.

8. The catheter and dressing assembly of claim 1 wherein the dressing is configured such that attachment of the dressing's adhesive plate around the catheter-skin insertion site creates a first circumferential seal and joinder of the dressing and the catheter hub base creates a second circumferential seal.

9. The catheter and dressing assembly of claim 1 wherein the dressing body contains a re-sealable access portal or window that allows adjustment of the internal milieu of the sterile chamber formed by the sealing catheter-dressing-skin interaction.

10. The catheter and dressing assembly of claim 1, whereby the hub base has a secondary mating and catheter stabilization point consisting of a stop flange of a greater diameter than the remainder of the hub base, so that this stop flange mechanically prevents withdrawal of the catheter from the dressing once the dressing is mounted and mated with the catheter hub base.

11. The catheter and dressing assembly of claim 1 wherein at least one of the mating surfaces of the hub interconnector cap and hub base include an anti-microbial agent so that the joining of the interconnector cap to the hub base is rendered sterile.

12. A method of using the catheter and dressing assembly of claim 1 comprising:
inserting a portion of the catheter body into a blood vessel at an insertion site such that a distal end of the catheter is positioned at an appropriate position in the blood vessel;
connecting the interconnector-cap with a catheter hub of a catheter;
sliding the dressing along interconnector-cap;
applying the adhesive plate to patient skin surrounding the insertion site to create a first circumferential seal; and
joining a proximal portion of the dressing to a mating feature of the hub base or the interconnector-cap to provide a second circumferential seal around the catheter body.

13. The catheter and dressing assembly of claim 1, further comprising a flushing syringe configured to flush the newly inserted catheter and act as a mounting handle over which a sterile sealing dressing can be applied to a catheter insertion site.

14. The catheter and dressing assembly of claim 13, further comprising a peel-away sheath wherein the catheter is configured to be inserted into a blood vessel using a peel-away sheath insertion technique, whereby the catheter hub base and catheter interconnector cap complex is inserted through the previously positioned peel away sheath and into final position.

15. The catheter and dressing assembly of claim 14, wherein the catheter hub base, catheter hub interconnection cap, and a flush syringe/mounting handle are preassembled as a single reversibly interconnected unit that can be inserted into the patient using through a peel-away sheath technique.

16. The catheter and dressing assembly of claim 13, wherein an insertion wire comprises an integral part of the flush syringe.

17. A method of inserting the catheter and dressing assembly of claim 13, whereby the tubular portion of the catheter, the hub base, the hub interconnector cap, and the attached syringe are be passed over an insertion wire disposed within a blood vessel such that a distal end of the catheter is positioned at an appropriate position in the blood vessel.

18. The method of claim 17 further comprising sliding the dressing along the syringe and interconnector-cap; applying the adhesive plate to patient skin surrounding the insertion site to create a first circumferential seal; and joining a proximal portion of the dressing to a mating feature of the hub base or the interconnector-cap to provide a second circumferential seal around the catheter body.

19. The method of claim 17 whereby an accessory form fitting activity cover is attached as a protective form-fitting cover over the entire catheter-dressing complex.
